(19) United States
(12) Patent Application Publication
(10) Pub. No.: US 2005/0261335 A1
(43) Pub. Date: Nov. 24, 2005

(54) Carvedilol formulations

(76) Inventors: Wei Chen, Oaks, PA (US); Kimberly A. Lamey, King of Prussia, PA (US); Jennifer Malofiy, Oaks, PA (US); Choon Oh, Chester Springs, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/513,235

(22) PCT Filed: May 2, 2003
(86) PCT No.: PCT/US03/14020

§ 371 Date: Nov. 2, 2004

Related U.S. Application Data
(60) Provisional application No. 60/377,569, filed on May 3, 2002.
(60) Provisional application No. 60/379,221, filed on May 9, 2002.

(51) Int. Cl. A01N 43/38; A01N 43/42; A61K 31/47; A61K 31/40

(52) U.S. Cl. 514/310

(57) ABSTRACT

This invention relates to pharmaceutical formulations of carvedilol in which a solubilising agent is used to solubilise carvedilol in oils and/or lipids, and methods of of using these formulations to treat hypertension, congestive heart failure and angina.

### FIELD OF THE INVENTION

The present invention relates to novel formulations of carvedilol, and to the use of the formulations in therapy.

### BACKGROUND OF THE INVENTION

The compound, 1-(Carbazol-4-yloxy-3-[[2-(o-methoxyphenoxy)ethyl]-amino]-2-propanol is known as carvedilol. This compound has the following structure: 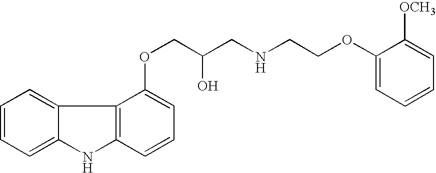
and is claimed in U.S. Pat. No. 4,503,067 (assigned to Boehringer Mannheim, GmbH, Mannheim-Waldhof, Fed. Rep. of Germany), issued Mar. 5, 1985.

Carvedilol is currently synthesized as free base for incorporation in medication that is available commercially. It is a racemic mixture of the R(+) and S(−) enantiomers, where nonselective β-adrenoreceptor blocking activity is present in the S(−) enantiomer and α-adrenergic blocking activity is present in both R(+) and S(−) enantiomers. This unique feature contributes to the two complementary pharmacologic actions: mixed venous and arterial vasodilation and non-cardioselective, beta- adrenergic blockade.

Carvedilol is used for treatment of hypertension, congestive heart failure and angina. The currently available product is a conventional, tablet prescribed as a twice-a-day medication in the United States.

Carvedilol contains an a-hydroxyl secondary amine, with a pKa of 7.8. It exhibits predictable solubility behaviour in neutral or alkaline media, i.e. above pH 9.0, the solubility is relatively low (<1 μg/mL). Its solubility increases with decreasing pH and reaches a plateau near pH 5: i.e. saturation solubility is ca 23 μg/mL at pH 7 and ca 100 μg/mL at pH 5 at room temperature. At lower pH values (pH 1 to 4 in buffer systems), solubility is limited by the solubility of the protonated form of carvedilol or its salt formed in-situ. The hydrochloride salt generated in-situ in an acidic medium, such as simulated gastric fluid, is less soluble in this medium than the protonated carvedilol itself.

There is a need for a carvedilol formulation that provides for prolonged drug levels in the systemic system by sustaining absorption along the gastro-intestinal tract.

Surprisingly, it has now been found that such novel formulations of carvedilol can be prepared.

### SUMMARY OF THE INVENTION

The present invention provides novel formulations of carvedilol.

The present invention also provides the use of these carvedilol formulation in the treatment of hypertension, congestive heart failure and angina.

### DETAILED DESCRIPTION OF THE INVENTION

In accordance with the present invention, it has been unexpectedly found that carvedilol can be formulated with solubility enhancers for increased absorption.

Carvedilol is claimed in U.S. Pat. No. 4,503,067 (the '067 patent). Reference should be made to said patent for its full disclosure, including the methods of preparing and using this compound. The entire disclosure of the '067 patent is incorporated herein by reference.

The present invention relates to novel formulations of carvedilol in the presence of solubility enhancers for increased dissolution in-vitro and absorption in-vivo. In particular, this invention is concerned with the use of Pharmasolve® in conjunction with structurally similar pharmaceutical excipients, such as, but not limited to, pyrrolidone, poly(ethylene oxide), poly(propylene oxide), poly(ethylene oxide)-poly(propylene oxide) copolymer, poly(ethylene oxide)-poly(propylene oxide)-poly(ethylene oxide) triblock copolymer. Pharmaceutical applications using such systems are drug layered beads, granulations, solutions, ointments, creams and suspensions. Forms of delivery may be orally, topically, rectally, vaginally, transdermally, intravenous, bolus injections or inhalation routes.

Pharmasolve®, N-methyl-2-pyrrolidone, is currently manufactured by International Specialty Products and is deemed a broad spectrum drug solubilizer for pre-clinical evaluation and dosage forms. It has been shown to increase the solubility, solubilization rate and drug stability in aqueous solution.

Structurally similar pharmaceutical excipients, such as polyvinyl pyrrolidone (PVP), N-vinyl-2-pyrrolidone/vinyl acetate (Copolyvidonum—Plasdone® S-630) and poloxamer, have also shown to increase the solubility of poorly soluble pharmaceutically active compounds, such as carvedilol. These ingredients in combination with Pharmasolve® may potentially increase solubility, while providing enhanced stability on storage.

The formulation may consist of a liquid-solid granulation filled into capsules and subsequently film coated or a liquid-solid granulation either blended with or without external excipients, tableted and then subsequently film coated. These formulations will include Pharmasolve® in combination with a structurally similar pharmaceutical ingredient. The liquid-solid granulation may be prepared, for example, as described in U.S. Pat. No. 4,719,228, issued Jan. 12, 1988 and U.S. Pat. No. 4,859,709, issued Aug. 22, 1989.

The formulation may also consist of carvedilol dissolved in a carrier which is subsequently filled into capsules as either liquid or solid. The Pharmasolve®, structurally similar pharmaceutical ingredient and carvedilol, along with other carriers or excipients may be prepared as described in PCI application WO 99/26625, published Jun. 3, 1999.

Typically, the capsule comprises a capsule shell containing carvedilol as the free base or a pharmaceutically acceptable salt or solvate thereof in solution in a carrier. The carrier may be liquid or solid.

By the term “pharmaceutically acceptable salt” is meant an acid addition salt of carvedilol, prepared in a standard manner in a suitable solvent from the parent compound and an excess of an acid, such as hydrochloric, hydrobromic, hydrofluoric, sulfuric, phosphoric, acetic, trifluoroacetic, benzoic, citric, maleic, succinic or methanesulfonic.

A liquid carrier may be a solvent present in the capsule as a flowable liquid, as a viscous liquid or semi-solid or as a gel. The carrier may also be a solid or semi-solid solvent such as fats and waxes, or film-forming or thermoplastic polymers. Solvents in which supersaturated solutions can be formed are advantageous because of the possibility to increase the loading of active ingredient.

When the carrier is a solid or semi-solid or a gel, the calvedilol containing carrier may be self-supporting without encapsulation. Accordingly a self-supporting formulation may be encapsulated by other means than loading into a preformed capsule shell, for example by coating with an encapsulating material. Also the self-supporting formulation may be used as a dosage form without encapsulation.

Accordingly in another aspect the present invention provides an oral swallow solid dosage form containing carvedilol dissolved in a solid, semi-solid or gel carrier.

Typically the solid dosage form comprises tablets, pellets, spheroids, granules, lozenges or gels in which carvedilol is present as a solid solution in a polymeric carrier.

Capsules and solid dosage forms of this invention may be coated to assist in administration of the active ingredient, for example using an enteric coating material to prevent release of carvedilol in the stomach, coatings to delay or control release of carvedilol and coatings of taste-maslcing agents. Alternatively, such materials can be incorporated in the carrier to achieve the same effect.

The amount of carvedilol used in each capsule is preferably adjusted such that in a single unit dose there is a therapeutically effective amount of carvedilol. Suitably, the unit dose contains from 3.125 to 50 mg carvedilol, preferably, 50 mg of carvedilol, given once or twice daily, preferably given once daily.

To achieve the desired unit dose in a capsule where the carvedilol is in solution in the carrier, the carvedilol needs to be soluble in the carrier to an extent that allows a sufficient concentration so that the selected capsule volume can contain the desired unit dose. In addition to being able to dissolve carvedilol, the solvent must be compatible with the capsule material and physiologically acceptable for administration to a patient.

Suitable capsules of the instant invention have a maximum volume of 0.86 ml. Preferred capsules according to the present invention have a maximum volume of about 0.45 ml and more especially may lie in the range 0.2 to 0.4 ml, although capsules as small as 0.14 ml are also provided by the invention. A typical capsule at the upper end of the size range acceptable for pharmaceutical use (Soft Gel Size 14 Oblong) has a volume of 0.86 ml.

The present inventors have found that an especially effective means to solubilise carvedilol in a liquid, semi-solid or solid carrier, in particular oils and lipids, is to use a solubilising agent, such as N-methyl-2-pyrrolidone (Pharmasolve, International Speciality Products, Texas, USA) as a cosolvent.

Accordingly in a preferred embodiment of this invention, carvedilol, optionally as the free base or as a pharmaceutically acceptable salt, is dissolved in a solubilising agent and then blended with an oil or lipid carrier before filling capsules.

The invention also provides as a novel formulation a solution of carvedilol, optionally as the free base or as a pharmaceutically acceptable salt in a blend of a solubilising agent and a lipid and/or oil.

The capsule shell may be of any conventional material that is stable to the liquid carrier and solute, for example hard and soft gelatin capsules and starch capsules. In addition to resisting the solvent action of the liquid carrier attention must be paid to the pH of the liquid within the capsule. For example soft gels have a pH limit of 2.5-7.5.

According to a further aspect of the invention, the capsules have an enteric resistant coating or incorporate enteric resistant materials in the capsule shell, such that carvedilol is not discharged in the acidic conditions of the stomach. The object of this is to prevent any undesired uncontrolled precipitation of the carvedilol from solution, and to enable its absorption characteristics to be modified if desired by presenting it to the intestinal mucosa in non-aqueous solution.

The liquid carrier may be present in the capsule as a flowable liquid, as a viscous liquid or semi-solid or as a gel. The viscosity characteristics may be varied by initial choice of solvent or by appropriate use of cosolvents or thickening agents.

A liquid carrier, or a solid or semi-solid carrier that has been softened or made flowable by heating, with dissolved carvedilol may be filled into capsules using conventional capsulation technology.

The present invention also provides solid dosage forms of carvedilol for oral swallow use in which carvedilol is dissolved in a polymeric carrier. These forms include tablets, pellets, spheroids, granules, lozenges and gels containing carvedilol in solid solution.

To achieve the desired unit dose in, for example, a melt extruded tablet where carvedilol is in solution in the polymer carrier, carvedilol needs to be soluble in the polymer carrier or a solvent/cosolvent that is soluble in the polymer carrier to an extent that allows a sufficient concentration so that the selected tablet size and volume can contain the desired unit dose. In addition to being able to dissolve carvedilol, the solvent/cosolvent must be compatible with the polymer carrier material and physiologically acceptable for administration to a patient.

When the solid dosage form is granules or pellets then a plurality of granules or pellets may be collected in an aggregation that as a whole constitutes a unit dose. The granules or pellets may be used as a fill for capsules or pressed, optionally with binders or excipients, into tablet form.

In general the use of polymers in this invention to produce semi-solid/solid solution system offer a broad flexibility of use. Beside filling into hard/soft gelatin capsules they may be used to make melt extruded system such as tablets, pellets, spheroid and any other shape depending on the shape of the extruder die, can be injection moulded into different shapes and/or melt granulated to produce pellets or granules. Alternatively the granules can be milled and pressed into tablets and other shapes depending on the shape and design of the press die.

According to a further aspect of the invention, the solid dosage form may have an enteric resistant coating such that carvedilol is not discharged in the acidic conditions of the stomach. The object of this is to prevent any undesired uncontrolled precipitation of the carvedilol from solution, and to enable its absorption characteristics to be modified if desired by presenting it to the intestinal mucosa in an aqueous solution.

The semi-solid or gel formulation can also be optionally capsulated. The viscosity characteristics of the semi-solid or gel may be varied by initial choice and amount of solvent or by appropriate use of cosolvents or thickening agents.

The semi-solid or gel carrier with dissolved carvedilol may be filled into capsules using conventional encapsulation technology.

Self-supporting solid of carvedilol solution can be successfully prepared in forms of tablet, pellets, spheroid, granules using Solan E, Gelucire, higher molecular weights of PEG's and gel based on gelatin with different cosolvents constituents.

Therapeutic uses of the carvedilol formulations of this invention include the treatment of: congestive heart failure, hypertension and angina.

### EXAMPLES

The invention is illustrated by the following Examples:

Formulations were prepared as liquid-solid granulations using Pharmasolve® and either polyvinyl pyrrolidone (PVP) or Plasdone® S-630 (produced by International Speciality Products) by the following method:
1. Follow standard procedures for using either a fluid-bed granulator or high shear wet granulator. The liquid-solid granulation does not require drying as to protect the integrity of the amount of Pharmasolve® (solvent) included in the batch during preparation.
2. Weigh appropriate amounts of the desired ingredients to prepare the spray solution. Heat the Pharmasolve® and structurally similar pharmaceutical excipient to 30° C.-35° C.
3. Slowly add the pharmaceutically active compound to the Pharmasolve® mixture making sure that all material is dissolved prior to granulating.
4. Weigh separate quantities of bulking excipients to prepare the batch and add to the granulator bowl.
5. For a fluid-bed granulation batch, prepare a pump spray to deliver appropriate amounts of solution onto the bulking ingredients. Maintain a product temperature of 35° C. or below while granulating and stop the process upon complete addition of the solution.
6. For a high shear granulation batch, mix the bulking excipients for 3 minutes with an appropriate impeller speed and chopper set point. Tare the balance prior to pouring the solution/suspension onto the bulking ingredients in the high shear granulator at a constant rate. Start processing by monitoring solution/suspension addition, impeller and chopper set points and the material characteristics.
7. Upon reaching the granulation end-point, sieve the liquid-solid granulation through a #12-18 mesh screen. Measure the moisture content to determine if the desired amount of Pharmasolve is still included in the granulation batch.

Examples of liquid-solid granulations are shown below:

### EXAMPLE 1

### Drug: Pharmasolve: Structurally Similar Excipient (1:2:0.5 Ratio)

| | |
|---|---|
| | Master Formula Quantity |
| Ingredient | (% w/w) |

Spray/Pour Solution

| | |
|---|---|
| Carvedilol | 9.6 |
| Pharmasolve ® | 19.2 |
| PVP or Plasdone S-630 | 4.8 |
| Bulking Solid Ingredients | |
| Crospovidone | 4.0 |
| Microcrystalline Cellulose (Avicel PH-102) | 62.4 |
| Total | 100.0 |

### EXAMPLE 2

### Drug: Pharmasolve: Structurally Similar Excipient (1:2:1 Ratio)

| | |
|---|---|
| | Master Formula Quantity |
| Ingredient | (% w/w) |

Spray/Pour Solution

| | |
|---|---|
| Carvedilol | 9.6 |
| Pharmasolve ® | 19.2 |
| PVP or Plasdone S-630 | 4.8 |
| Bulking Solid Ingredients | |
| PVP or Plasdone S-630 | 4.8 |
| Crospovidone | 4.0 |
| Microcrystalline Cellulose (Avicel PH-102) | 57.6 |
| Total | 100.0 |

### EXAMPLE 3

### Drug: Pharmasolve: Structurally Similar Excipient (1:2:1.5 Ratio)

| | |
|---|---|
| | Master Formula Quantity |
| Ingredient | (% w/w) |

Spray/Pour Solution

| | |
|---|---|
| Carvedilol | 9.6 |
| Pharmasolve ® | 19.2 |
| PVP or Plasdone S-630 | 4.8 |
| Bulking Solid Ingredients | |
| PVP or Plasdone S-630 | 9.6 |
| Crospovidone | 4.0 |
| Microcrystalline Cellulose (Avicel PH-102) | 52.8 |
| Total | 100.0 |

### EXAMPLE 4

### Drug: Pharmasolve: Structurally Similar Excipient (1:2:2 Ratio)

| | |
|---|---|
| | Master Formula Quantity |
| Ingredient | (% w/w) |

Spray/Pour Solution

| | |
|---|---|
| Carvedilol | 9.6 |
| Pharmasolve ® | 19.2 |
| PVP or Plasdone S-630 | 4.8 |
| Bulking Solid Ingredients | |
| PVP or Plasdone S-630 | 14.4 |
| Crospovidone | 4.0 |
| Microcrystalline Cellulose (Avicel PH-102) | 48.0 |
| Total | 100.0 |

Compaction properties of the liquid-solid granulations were measured using a Mand compaction simulator equipped with standard 10 mm flat-faced tooling. It was determined that higher levels of structurally similar pharmaceutical excipients included in the granulation provided significantly higher tablet strength and increased ductility. Addition of external bulking agents post granulation is recommended for robust solid oral dosage manufacture. It has also been shown that drying after high shear or fluid-bed granulation to produce a more solid system is acceptable.

Solubilization Capabilities

To ascertain the importance of including a structurally similar pharmaceutical excipient in combination with Pharmasolve® and carvedilol, a poorly soluble compound at neutral pH, assay for percent label claim and solubility testing as dissolution, Apparatus 2 (paddles at 50 RPM), using 500 ml 0.05M sodium phosphate buffer, pH 6.8, without sodium dodecyl sulfate was performed. Coreg commercial compression mix is used as control for comparison purposes.

Assay:

| | Granulation | Assay | % Label |
|---|---|---|---|
| Batch | (Ratio) | (% w/w) | Claim |
| 111 | Wet, A (1:2:1) | 9.19 | 95.7 |
| 107 | Wet, B (1:2:1.5) | 9.22 | 96.0 |
| 112 | Wet, C (1:2:2) | 9.16 | 95.4 |
| 010 | Coreg Commercial | 8.27 | 99.3 |

Dissolution:

| Procedure: ˜100 mg Carvedilol | |
|---|---|
| pH data: pH of media = 6.8 | |
| pH of media after 4 h: | Batch 111 = 6.78 |
| | Batch 107 = 6.78 |
| | Batch 112 = 6.79 |
| | Batch 010 = 6.75 |

TABLE 1
Solubility (ug/ml) during dissolution testing.

| | | | Solubility (ug/ml) | | | | | |
|---|---|---|---|---|---|---|---|---|
| Batch | Granulation (Ratio) | Time (min) | 15 | 30 | 60 | 120 | 180 | 240 |
| 111 | Wet, A (1:2:1) | Average | 105 | 120 | 131 | 138 | 141 | 143 |
| | | n = 2 | | | | | | |
| 107 | Wet, B (1:2:1.5) | Average | 101 | 119 | 131 | 139 | 144 | 146 |
| | | n = 2 | | | | | | |
| 112 | Wet, C (1:2:2) | Average | 101 | 118 | 133 | 143 | 149 | 152 |
| | | n = 2 | | | | | | |
| 010 | Carvedilol | Average | 29 | 31 | 34 | 36 | 37 | 36 |
| | Commercial | n = 2 | | | | | | |
| | Compression Mix | | | | | | | |

From solubility studies, it was determined that the inclusion of Pharmasolve® and structurally similar pharmaceutical excipients in a formulation containing a poorly soluble compound, such as carvedilol, provides increased solubility in-vitro at pH 6.8.

In-Vivo Animal Testing

Three, fasted beagle dogs were dosed with a 10 mg dose of Carvedilol via an InteliSite® Companion capsule radiolabeled with Indium-111. The capsules were dosed with approximately 20 mL of water. The radiolabel served to track position of the capsule in the gastrointestinal tract, and to verify that it had released its contents. Opening of the capsule occurred after passing the duodenal loop and served as time “zero” for blood sampling.

The Pharmasolve® granulation was prepared by dissolving desired amounts of Carvedilol and Plasdone S-630 into Pharmasolve®, N-methyl-2-pyrrolidone, solvent. Bulking ingredients were mixed for three minutes in a small scale granulator. The Carvedilol, Plasdone S-630 and Pharmasolve® mixture was then slowly poured over the bulking ingredients while measuring chopper and impeller speed over a time of 3 minutes. Upon completion of mixture addition, the ingredients were mixed for another 20 minute. The completed granulation was not dried in order to form a liquid:solid final product, and the loss on drying measurement resulted in a 19.09% moisture content (i.e. primarily as a result of solvent loss during testing). To accommodate a 10 mg dose in the InteliSite® Companion capsule, 104.2 mg granulation was required per capsule. Formulation composition is shown in Table 2.

TABLE 2
Carvedilol Pharmasolve ® Granulation Composition

| | Unit |
|---|---|
| | Formula |
| Formulation Composition | (% w/w) |
| Pour Solution | |
| Carvedilol | 9.6 |
| Pharmasolve ® | 19.2 |
| Plasdone S-630 | 4.8 |
| Bulking Ingredients | |
| Plasdone S-630 | 14.4 |
| Crospovidone, (Polyplasdone XL10) | 4.0 |
| Avicel PH-102 | 48.0 |
| Total | 100.0 |

Coreg commercial compression mix was used as a control to test the hypothesis whether formulations that exhibit enhanced solubility at pH 6.8 deliver enhanced exposure when released in the lower part of the GI tract.

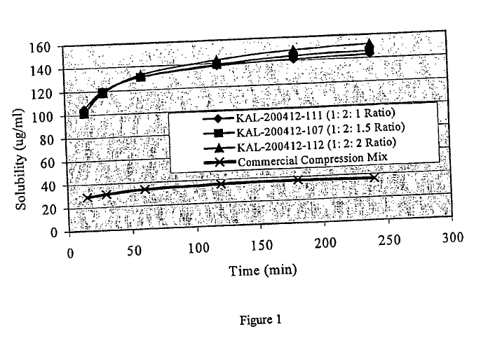

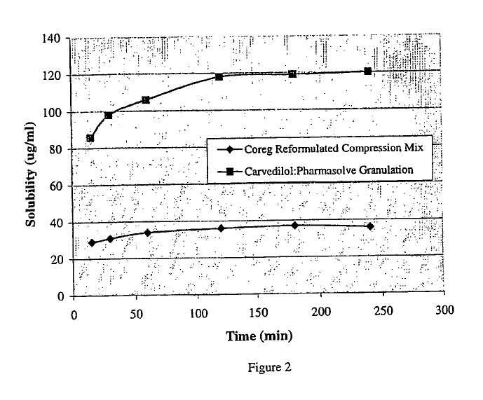

Formulation solubility was performed using enough formulation to equate to 100 mg carvedilol. The material was dispersed in 500 ml 0.05M phosphate buffer, pH 6.8 in Apparatus 2 (paddles at 50 RPM). Solubility was measured at various timepoints as shown in Table 3 and FIG. 2.

TABLE 3
Carvedilol Formulation Solubility
(ug/ml) in 0.05 M Phosphate Buffer

| | | Time (min) | | | | | |
|---|---|---|---|---|---|---|---|
| Formulation | Batch | 15 | 30 | 60 | 120 | 180 | 240 |
| Coreg Compression Mix | 010 | 29 | 31 | 34 | 36 | 37 | 36 |
| Carvedilol: | 037 | 86 | 98 | 106 | 118 | 119 | 120 |
| Pharmasolve ® | | | | | | | |
| Granulation | | | | | | | |

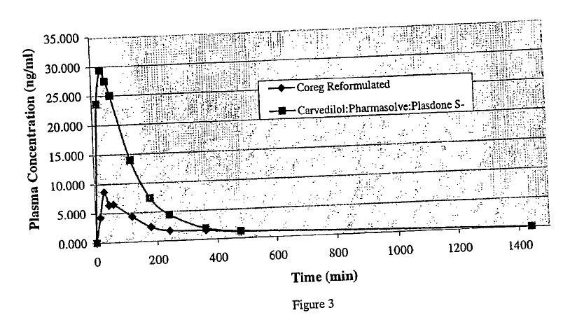

Subsequent pharmacoltinetic data was generated and is shown in Table 4 and FIG. 3.

TABLE 4
Pharmacokinetic analysis of 10 mg carvedilol dosed
formulations in three fasted beagle dogs.

| | Cₘₐₓ | Tₘₐₓ | AUC (0 − t) |
|---|---|---|---|
| Formulation | (ng/mL) | (min) | (ug.min/mL) |
| 10 mg Carvedilol | 31.32 ± 3.43 | 15, 30, 45 | 4.03 ± 1.34 |
| Liquid/Solid Granula- | (n = 3) | (n = 3) | (n = 3) |
| tion at a 2:2:1 Ratio | | | |
| (Pharmasolve:Plasdone | | | |
| S630:Carvedilol) | | | |
| 10 mg Coreg Reform- | 13.08, 12.74, 2.89 | 45, 30, 120 | 2.14, 1.19, |
| ulated IR Compression | (n = 3) | (n = 3) | 0.60 (n = 3) |
| Mixture | | | |

Surprisingly, it was shown that a formulation containing Pharmasolve®, a structurally similar pharmaceutical excipient and carvedilol not only increased absorption in the lower part of the GI tract, but also decreased inter-animal variability. Comparison of the in-vivo PK data versus in-vitro solubility data for the Pharmasolve® formulation surprisingly supported the hypothesis that enhanced carvedilol solubility at pH 6.8 results in enhanced carvedilol exposure when released in the lower part of the GI tract. This invention is preferable by oral administration, but not necessarily limited by oral application.

It is to be understood that the invention is not limited to the embodiments illustrated hereinabove and the right is reserved to the illustrated embodiments and all modifications coming within the scope of the following claims.

The various references to journals, patents, and other publications which are cited herein comprise the state of the art and are incorporated herein by reference as though fully set forth.

1. A pharmaceutical formulation comprising carvedilol or a pharmaceutically acceptable salt thereof in the presence of a solubility enhancer.

2. The formulation according to claim 1 wherein the solubility enhancer is Pharmasolve®.

3. The formulation according to claim 2 further comprising a structurally similar pharmaceutical excipient.

4. The formulation according to claim 3 wherein the structurally similar pharmaceutical excipient is polyvinyl pyrrolidone or N-vinyl-2-pyrrolidone/vinyl acetate.

5. The formulation according to claim 2 wherein the ratio of carvedilol to Pharmasolve® is 1:2.

6. A pharmaceutical formulation comprising 0.5%-10% (w/w) carvedilol, 1%-20% (w/w) Pharmasolve®, and 0.25%-20% (w/w) a structurally similar pharmaceutical excipient.

7. The formulation according to claim 6 comprising 5%-10% (w/w) carvedilol, 10%-20% (w/w) Pharmasolve®, and 2.5%-20% (w/w) a structurally similar pharmaceutical excipient.

8. The formulation according to claim 7 wherein the structurally similar pharmaceutical excipient is polyvinyl pyrrolidone or N-vinyl-2-pyrrolidone/vinyl acetate.

9. A method of treating hypertension, congestive heart failure or angina which comprises administering to a subject in need thereof an effective amount of the formulation according to claim 1.
